(19) United States
(12) Patent Application Publication
(10) Pub. No.: US 2009/0226934 A1
(43) Pub. Date: Sep. 10, 2009

(54) PROCESS FOR THE DETECTION OF ANALYTES

(76) Inventor: Jeffery C. Hill, Westminster, CO (US)

Correspondence Address: POLSINELLI SHUGHART PC, 700 West 47th Street, Suite 1000, KANSAS CITY, MO, 64112, US

(21) Appl. No.: 12/398,144

(22) Filed: Mar. 4, 2009

Related U.S. Application Data
(60) Provisional application No. 61/033,516, filed on Mar. 4, 2008.

(51) Int. Cl. G01N 33/567; G01N 33/543

(52) U.S. Cl. 435/7.2; 436/518

(57) ABSTRACT

An improved process for the detection of analytes in a liquid, including pathogens and other toxic substances, using liquid crystal display technology comprising coating a test cell with one or more antibodies, flowing a test sample through the cell, rinsing the cell to remove any uncaptured analytes, introducing and aligning a liquid crystal in the test cell, and observing the liquid crystal for disruptions indicative of the presence of analytes in the test sample. If the analyte is smaller than the critical diameter required to disrupt the detection medium, an interferent rinse, which may include antibody-coated microspheres, may be flowed through the test cell.

### CROSS REFERENCE TO RELATED APPLICATIONS

This application claims the benefit of U.S. Provisional Application No. 61/033,516 filed Mar. 4, 2008, which is incorporated herein by reference in its entirety as if fully set forth herein.

### FIELD OF THE INVENTION

The present invention relates to the use of a liquid crystal detection technology for the purposes of detecting the presence of analytes in a liquid. More specifically, the present invention relates to a process for the detection of analytes in a liquid, including pathogens and other toxic substances, which exhibits significantly improved accuracy, sensitivity, speed and convenience of testing and the capability of testing for more than one analyte in a single test specimen.

### BACKGROUND OF THE INVENTION

Recent efforts to develop systems for detecting certain analytes and ligands such as pathogenic agents, microbes or toxic substances and other substances in drinking water supplies, food products or blood samples, have lead to new detection methodologies and devices which make use of the physical properties of liquid crystals to indicate the presence of an analyte in a liquid test specimen. Typically, such systems employ various immunological techniques on a molecular level to coat microspheres with known antibodies which attach to a pathogenic agent or other analyte in a manner such that the alignment of the liquid crystals in the liquid crystal matrix is disturbed. When cross polarizers are placed on the exterior, the disturbance in the alignment of the liquid crystal allows light to pass through the polarizers, thus generating a visible signal indicating the presence of a pathogen.

Devices and methods for detection of agents in liquids are disclosed in U.S. Pat. No. 6,171,802, issued Jan. 9, 2001 to Woolverton, et al., and U.S. Pat. No. 7,160,736, issued Jan. 9, 2007 to Niehaus, et al. These detection devices may be adapted for use in the field so that they may be carried to site locations where test samples may be extracted directly from bodies of water such as lakes, reservoirs and rivers, by way of example, and tested onsite. Testing devices of this type may employ cartridges containing the liquid crystal matrix and various antibodies.

A key element of the overall process involves charging the cartridges with the liquid crystal and thereafter aligning the liquid crystal prior to sample testing. The alignment times may consume on the order of 100 minutes or longer using presently known techniques. It is clear, therefore, that known alignment methodologies are overly time consuming, labor intensive and not conducive to high speed testing cycles such as might be encountered in epidemic or bio-terrorism situations where a relatively large number of test samples may be taken, processed and analyzed in a very short time frame.

Prior art testing processes such as those described above may also involve creating aggregates of pathogens and antibody-coated microspheres in a mixing vial. These aggregates are then delivered in a suitable liquid crystal medium, sodium cromolyn, by way of example, into a glass slide and aligned. The aggregates, which create distortions in the liquid crystal, are then detectable visually and capable of measurement. However, because the ratio of material in the mixing vial to the amount of material delivered to the viewing area is high (on the order of ten to one at a minimum); detectability requires that a large number of aggregates be present in the vial. Moreover, each individual pathogen tested must be in a cell separate from the cells of any other pathogen involved in the testing process, which necessitates dividing up the test sample into smaller separate samples as needed. Further, prior to aggregate formation, all particles larger than a predetermined critical diameter (approximately three microns) for the visual testing process must be removed.

In addition to the foregoing, the prior art processes discussed above are further limited by not having internal process controls within the test cell or chamber. Placement of a negative control (an antibody that is nonreactive with the known elements within a test sample) within the test chamber itself would enhance the ability to determine background noise and non-specific binding activity. Placement of a control antibody within the test chamber and the addition of a control analyte that is paired to the control antibody during a testing sequence can verify if a successful assay has occurred.

It can be appreciated that the prior art processes described above entail a complex series of steps such as filtration and concentration measurement, which must be followed carefully in order to obtain any degree of reasonable reliability in the measured test results. Under time pressures in the field, particularly under crisis situations such as those that would exist during an epidemic or under a terrorist threat, a less demanding, less expensive and yet more sensitive test procedure would be desired to attain reliable test results.

Accordingly, a need exists for a technique for testing for analytes, namely, pathogens, toxins and other forms of biohazardous materials using liquid crystal matrices in systems designed for the detection of the aforementioned agents. The methodology disclosed herein provides a testing process having significantly improved sensitivity, improved accuracy, reduced sample concentration and filtration requirements and elimination of the need to split the sample into separate testing cells.

### BRIEF DESCRIPTION OF THE FIGURES

FIG. 1 is a top and side cross sectional view of a flow cell in accordance with an embodiment of the present invention;

FIG. 2 is a top view of a portion of the flow cell of FIG. 1 illustrating the introduction of a test sample into the cell;

FIG. 3 is a top view of the flow cell of FIGS. 1 and 2 illustrating a rinse step in accordance with an embodiment;

FIG. 4 is a top view of the flow cell of FIGS. 1-3 illustrating the introduction of antibody—coated microspheres into the flow cell;

FIG. 5 is a top view of the flow cell of FIGS. 1-4 illustrating yet another rinse step in accordance with an embodiment;

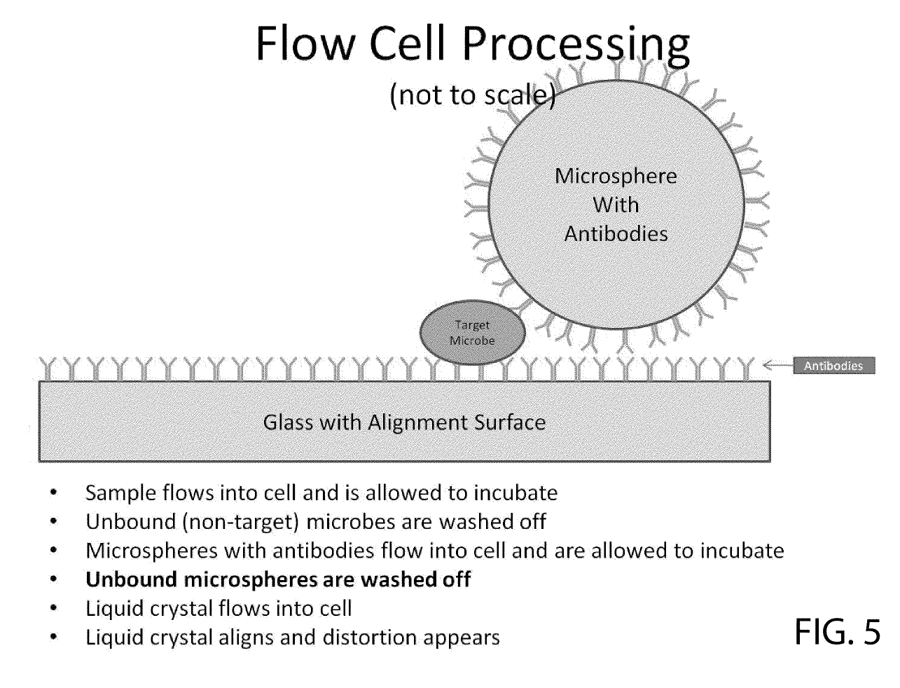

FIG. 6 is a top view of the flow cell of FIGS. 1-5 illustrating the introduction of liquid crystal into the flow cell in accordance with an embodiment; and

FIG. 7 is a top view of the flow cell of FIGS. 1-6 illustrating the alignment and distortion of the liquid crystal in accordance with an embodiment.

### DESCRIPTION OF THE INVENTION

Before proceeding with the detailed description, it should be noted that the present teaching is by way of example, not by limitation. The concepts presented herein are not limited to use or application with one specific type of analyte detection apparatus and methodology. Thus, although the instrumentalities described herein are for the convenience of illustration and explanation, shown and described with respect to exemplary embodiments, the principles disclosed herein may be applied to other types of analyte detectors and detection methods without departing from the scope of the present invention.

The improved process of the instant invention combines liquid crystal technology and flow cell methodology (or so-called “sandwich assay” testing methodology) to create an extremely sensitive, flexible, inexpensive and user-friendly pathogen detection system. Because the process includes wash steps, the need for sample filtration is reduced greatly and non-specific binding is reduced. The required levels of concentration in the specimens are achieved by flowing more material through the test cell.

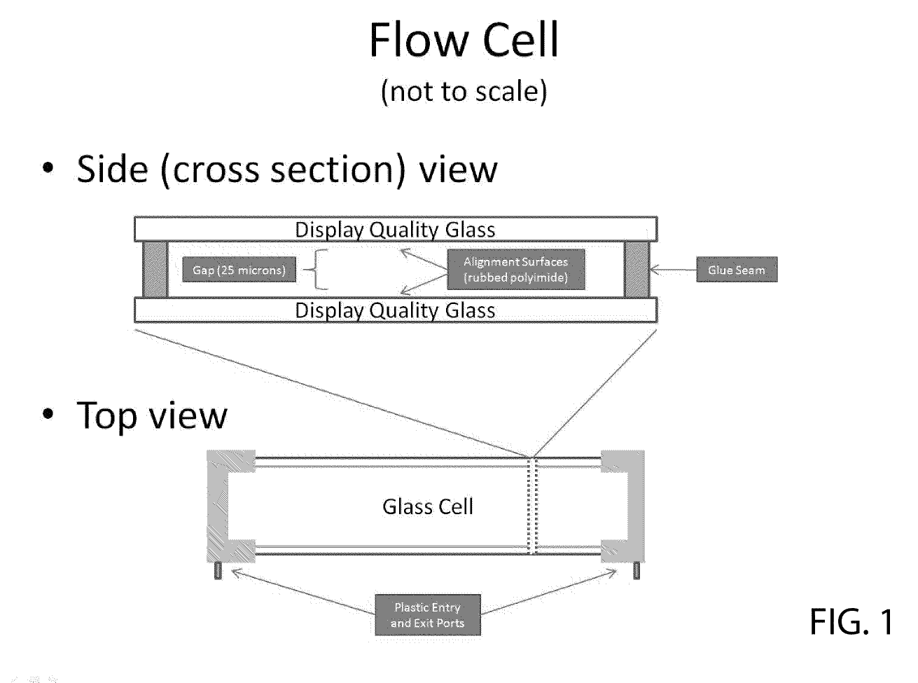

More specifically, a test sample containing pathogens is flowed directly through a flow or test cell, such as the flow cell shown in FIG. 1. The terms “test cell” and “flow cell” will be used interchangeably herein. One or both of the walls of the flow cell are coated with a capture antibody against an analyte, or, in a situation of testing for multiple analytes, patches of different antibodies in the same cell.

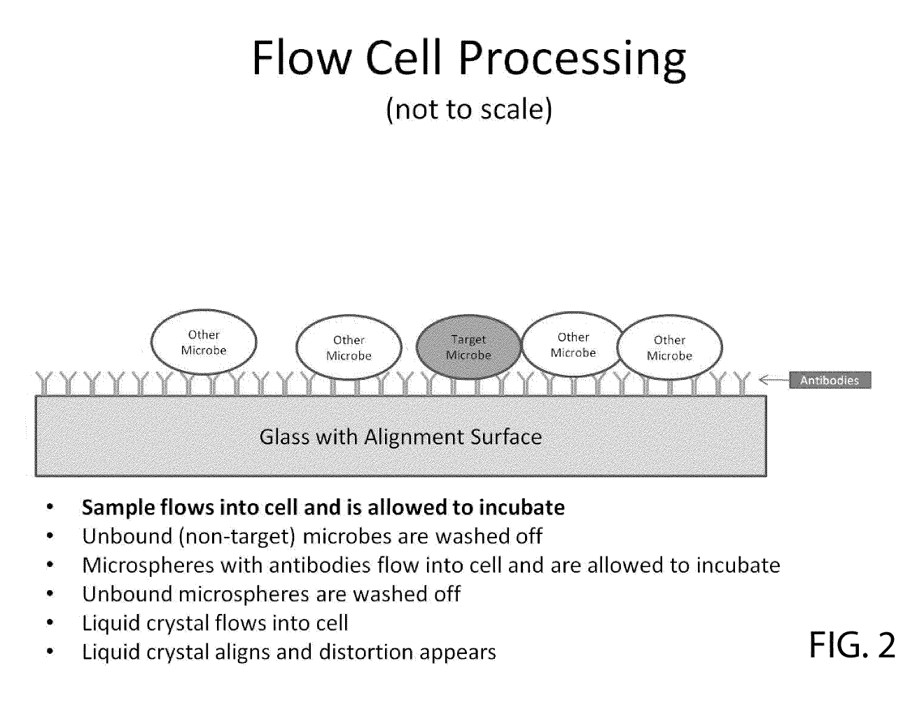

As illustrated more clearly in FIG. 2, the analytes bind to the appropriate antibody and are captured. The sensitivity and the accuracy of the detection process may be maximized by monitoring and controlling various process variables which include, but are not limited to: the test sample concentration, flow rate, temperature, salinity, and pH. Certain properties of the test sample, by way of example, salinity and pH, may be controlled via the introduction of buffering solutions containing detergents and/or surfactants, which also aid the rinse process in removing marginally bound and undesirable particles, unwanted analytes and contaminants from the cell. Adjusting the aforementioned variables, and, indeed, even stopping the test specimen flow through the cell for a selected period of time, for example, several seconds to ten minutes or longer, maximizes the effects of the incubation period, or the period of time in which the target analyte is in contact with an antibody.

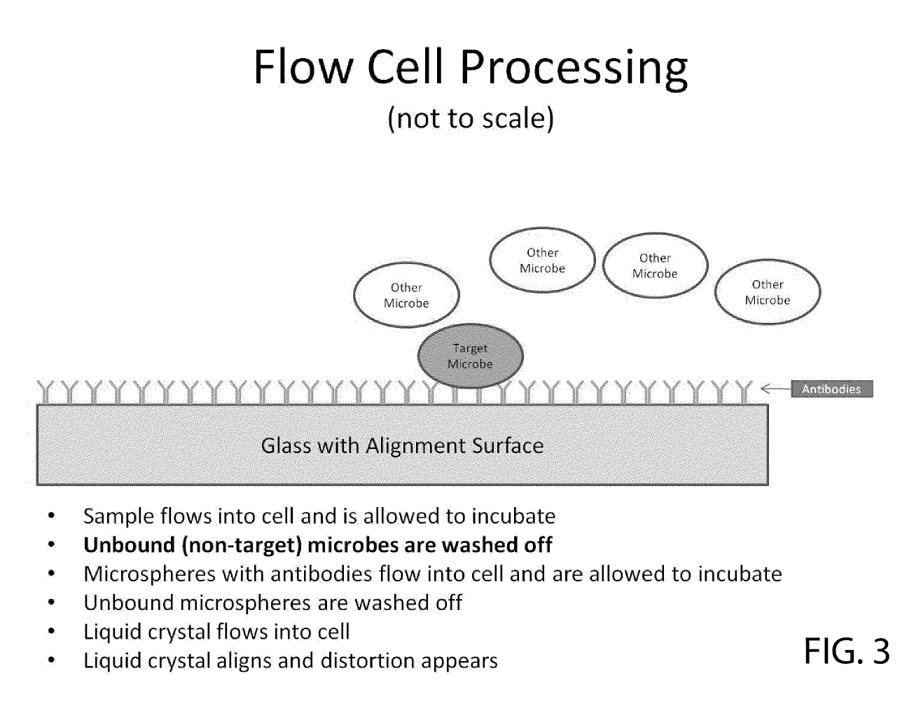

Referring now to FIG. 3, the test cell is then rinsed or washed by flowing a wash compound through the test cell to remove non-specifically bound analytes and other unwanted substances from the test cell. Non-specific binding occurs when an analyte or another particle, by way of example, a protein, which may have sticky surface characteristics, adhere to an antibody or even to the spaces on the test cell surfaces intermediate the antibodies. Such non-specifically bound particles may affect the accuracy of the detection process and should be minimized or eliminated to the extent possible. To this end, blocking agents may be added to the rinse to prevent non-specific binding, as well as buffering agents for controlling pH, salinity and other properties of the wash, as hereinabove described. Control antibodies and control analytes which are non-specific to the test sample may be added to control non-specific binding events and to verify the success of a particular test cycle, as discussed above. The rinse temperature may also be monitored and controlled to maximize its effectiveness in removing the non-bound elements and contaminants from the cell.

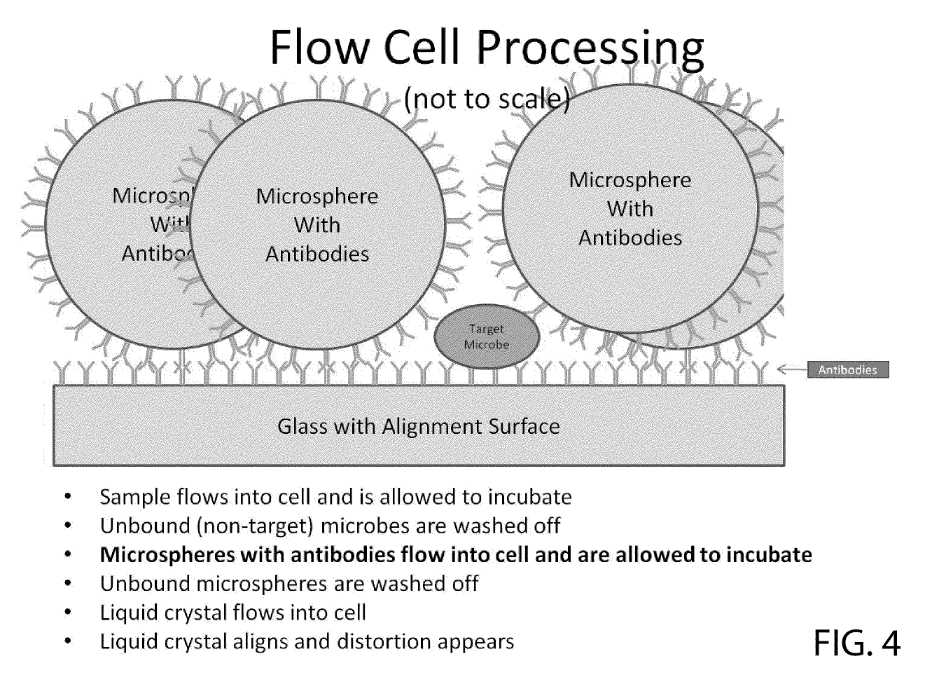

Referring now to FIG. 4, the next step in the detection process involves the application of a so-called sandwich technique wherein antibody-coated microspheres of varying preselected diameters and concentrations, depending upon the target analyte, are flowed through the test cell. The antibody-coated microspheres are allowed to incubate in the cell as described above with respect to the test sample, and will bind to the target pathogens or analytes already captured by the antibodies on the cell surfaces. The resulting analyte-microsphere complex is larger than the critical diameter needed to disrupt the detection medium, which will be introduced in a subsequent step. This sandwich step may be eliminated if the analyte or pathogen is larger than the critical diameter. The sandwich step may also be replaced by a chemical or other detection media interferent rinse or soak.

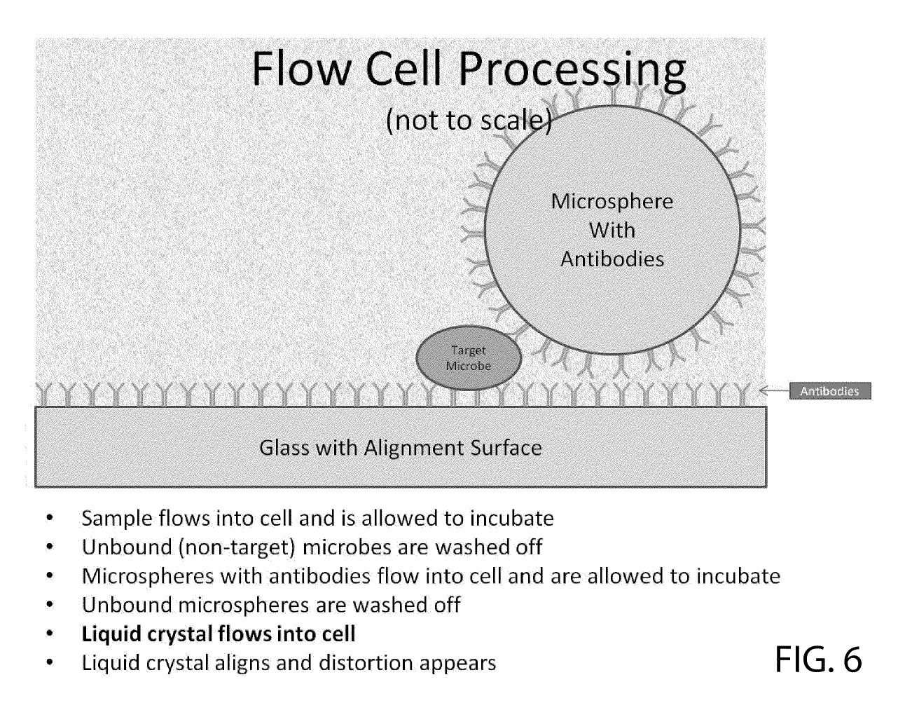

Any excess and/or unbound microspheres are then removed with a second rinse as illustrated in FIG. 6. This rinse cycle is monitored and controlling in the same manner as the initial rinse discussed above with respect to the test sample, i.e., flow rates, volumes, temperatures and buffering elements and blocking agents may also be selectively added to the rinse medium to maximize its effectiveness.

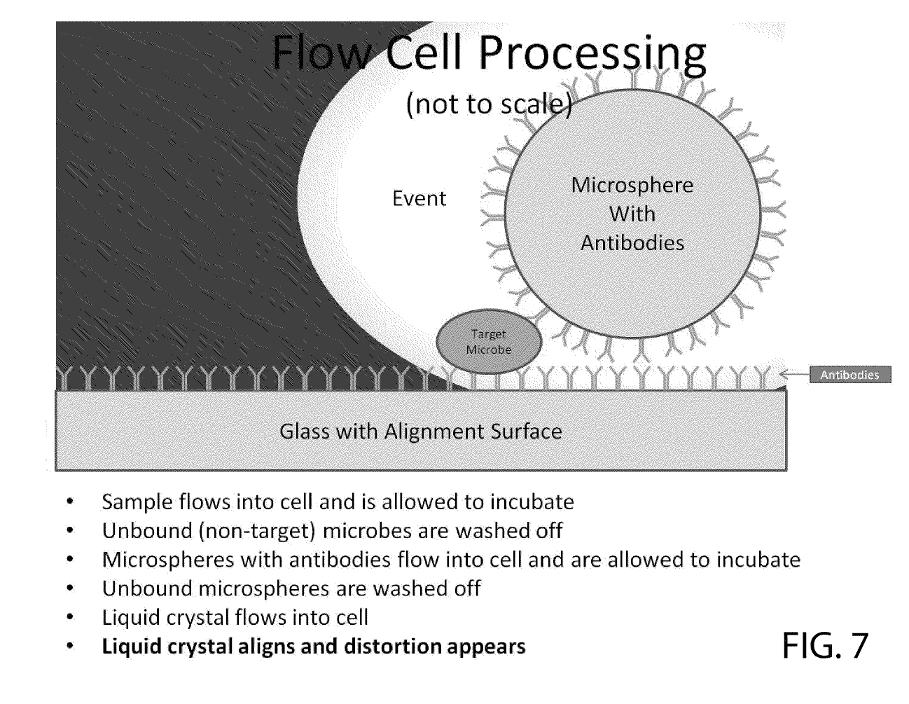

Following the microsphere rinse, the flow cell is filled with a detection medium as illustrated in FIG. 6, which, in an embodiment, comprises a liquid crystal of a suitable composition, by way of example, sodium cromolyn. The liquid crystal is then aligned. The detection process is carried out to determine the presence or absence of any of the target pathogens. The size of the bound analyte-microsphere structure is larger than the critical diameter required to disrupt the liquid crystal alignment, and polarized light directed toward the test cell is allowed to pass through the cell, thereby giving an indication of the presence of at least one analyte in the test specimen (FIG. 7).

Unlike the steps of the prior art processes, the test sample concentration is achieved by the capture of pathogens or other analytes by the antibodies bound to the slide substrate as the test sample is flowed through the cell. The wash step clears the test cell of any unbound particles, and the sample need only be filtered to remove any particles which are large enough to become physically stuck in the cell itself (10-30 microns). Since a single cell could contain patches of various antibodies, splitting the sample is no longer required, and the whole process becomes much more flexible, less time consuming and easier to read than prior art processes. Furthermore, internal positive and negative controls can be placed inside the chamber to improve overall assay performance.

Changes may be made to the above methods, systems and structures without departing from the scope of the present invention. It should be noted that the subject matter contained in the above description and/or shown in the accompanying drawings should be interpreted as illustrative and not in a limiting sense. The following claim(s) are intended to cover all generic and specific features described herein as well as statements of the scope of the present invention, which, as a matter of language, might be said to fall there between.

What is claimed is:

1. A process for the detection of analytes in a liquid comprising:
coating a test cell with at least one capture antibody;
flowing a test sample through the test cell;
rinsing the test cell to remove uncaptured analytes;
introducing a detection medium into the test cell; and
observing the detection medium for an indication of the presence of at least one analyte.

2. The process of claim 1 including the step of flowing interferent detection media through the test cell.

3. The process of claim 2 wherein the interferent detection media comprises antibody-coated microspheres.

4. The process of claim 3 including the step of rinsing the test cell to remove uncaptured microspheres.

5. The process of claim 1 wherein the detection medium comprises a liquid crystal and including the step of aligning the liquid crystal.

6. The process of claim 2 including the step of controlling the flow rate of the interferent detection media through the test cell.

7. The process of claim 1 including the step of controlling the flow rate of the test sample through the test cell.

8. The process of claim 1 including the step of controlling the incubation time of the test sample in the test cell.

9. The process of claim 1 including the step of adding blocking agents to the test sample.

10. The process of claim 1 including the step of adding surfactants to the test sample.

11. The process of claim 1 including the step of controlling the pH of the test sample in the test cell.

12. The process of claim 1 including the step of controlling the salinity of the test sample in the test cell.

13. The process of claim 1 including the step of controlling the temperature within the test cell.

14. The process of claim 3 including the step of controlling the diameters of the antibody-coated microspheres.

15. The process of claim 1 including the step of controlling the flow of the rinse to remove uncaptured analytes.

16. The process of claim 1 including the step of controlling the temperature of the rinse to remove uncaptured analytes.

17. The process of claim 1 including the step of controlling the pH of the rinse to remove uncaptured analytes.

18. The process of claim 1 including the step of adding blocking agents to the rinse to remove uncaptured analytes.

19. The process of claim 4 including the step of controlling the flow of the rinse to remove uncaptured microspheres.

20. The process of claim 4 including the step of controlling the temperature of the rinse to remove uncaptured microspheres.

21. The process of claim 4 including the step of controlling the pH of the rinse to remove uncaptured microspheres.

22. The process of claim 4 including the step of adding blocking agents to the rinse to remove uncaptured microspheres.

23. The process of claim 1 including the step of adding a control antibody to the test sample.

24. The process of claim 20 including the step of adding a control analyte to the test cell.
